(19) United States
(12) Patent Application Publication
(10) Pub. No.: US 2009/0093825 A1
(43) Pub. Date: Apr. 9, 2009

(54) System and Method for Providing a Coil Element in a Vascular Environment

(75) Inventor: John Logan, Plymouth, MN (US)

Correspondence Address: Patent Capital Group, 6119 McCommas Blvd, Dallas, TX, 75214, US

(73) Assignee: Castlewood Surgical, Inc.

(21) Appl. No.: 12/332,058

(22) Filed: Dec. 10, 2008

Related U.S. Application Data
(63) Continuation-in-part of application No. 11/842,541, filed on Aug. 21, 2007.
Application No. 11/842,541 is a continuation-in-part of application No. 12/273,484, filed on Nov. 18, 2008.

(51) Int. Cl. A61B 17/04; A61B 19/00

(52) U.S. Cl. 606/148; 128/898

(57) ABSTRACT

An apparatus is provided in one example and includes a coil that receives one or more tips of an instrument. An interior of the coil interfaces with the tips and the coil and the tips can be positioned together in a hole cut in a surface of tissue. The coil operable to be unwound such that a portion of the tips is revealed as the coil is unwound. In more specific embodiments, the instrument is a vein-holding apparatus, and the tips hold a vein to be sutured at the hole. In still other embodiments, the coil is helically shaped and includes a tail element that is pulled to unwind the coil incrementally. The tail element can be pulled until the tips are exposed from the coil.

### RELATED APPLICATIONS

This application claims priority under 35 U.S.C. §120 of pending U.S. application Ser. No. 11/842,541 and Entitled: System and Method for Providing an Obturator for Enhanced Directional Capabilities in a Vascular Environment, filed Aug. 21, 2007, and pending U.S. application Ser. No. 12/273,484 and Entitled: System and Method for Providing a Coil Element in a Vascular Environment, filed Nov. 18, 2008.

### TECHNICAL FIELD OF THE INVENTION

This invention relates in general to the field of cardiac and vascular surgery and, more particularly, to a process, a system, and a method for providing a coil element in a vascular environment.

### BACKGROUND OF THE INVENTION

In recent decades, the treatment of vascular diseases has grown exponentially in terms of sophistication and diversity. Most cardio-thoracic procedures, bypasses, and valve surgeries are routine, almost commonplace. Their popularity is due, in part, to their tremendous success rates and their ability to offer extraordinary benefits to a patient. Other types of surgeries have achieved a similar level of acceptance and popularity.

Many such procedures involve the use of medical devices, which have experienced considerable notoriety in recent years. Although these devices can automate and improve various types of procedures, many of these instruments do suffer from a number of significant drawbacks. For example, many medical devices include sharp points at their points of contact: points that generally snag or tear surrounding areas of tissue. In cases where an injury occurs, the surrounding tissue may be prone to inflammation, trauma, infection, or incomplete seals that can lead to bleeding and stroke. This detracts from the value of the surgery, adds unnecessary risk for a patient, and forces a surgeon to exercise extraordinary diligence in using such devices. Therefore, optimizing or simplifying any of these problematic issues may yield a significant reduction in risk for a patient and, further, minimize the accompanying burden for a surgeon.

Because a surgeon is generally tasked with estimating the approximate location of a target operating region [and in some cases, feeling his way through potential blind-spots], enhancing the accuracy of the placement of a given medical device would be highly beneficial and welcomed.

Accordingly, the ability to provide an effective medical tool that properly accounts for the aforementioned problems presents a significant challenge for component manufactures, system designers, and physicians alike.

### SUMMARY OF THE INVENTION

From the foregoing, it may be appreciated by those skilled in the art that a need has arisen for an improved instrument or tool for achieving superior control, management, and performance during a given procedure. In accordance with an embodiment of the present invention, a device, a system, and a method for enhancing an operation are provided that includes a flexible, precise, easy-to-use element, which substantially eliminates or greatly reduces disadvantages and problems associated with conventional equipment and instruments.

According to an embodiment of the present invention, an apparatus for assisting in a vascular procedure is provided that includes an obturator operable to be mounted on [or within] a medical device to guide the medical device into a targeted region, whereby the obturator is substantially blunt such that it exhibits a snag-resistant property.

In alternative embodiments, the apparatus includes an interface element operable to be used in conjunction with the obturator [or another device], whereby it can be used to engage an interface of the targeted region such that pressure is maintained within the targeted region. The interface element includes a convex portion that operates to seat the interface element at a selected location, which may be a round, oblong, or cut hole. The interface element is substantially transparent and includes a magnification element that magnifies materials underlying the interface element so as to see the underlying hole or incision. In other embodiments, the apparatus includes a tray operable to provide a mold for forming the obturator, which may comprise a gas, water, sugar, clotted blood, a gelatinous material, a protein, a saline solution, and dry-ice.

In specific embodiments, the obturator is operable to be used in conjunction with a medical device that includes one or more sharp or pointed legs. The legs of the medical device each include a tip that is sharp and operable to pierce the obturator such that it can be secured and transported.

Certain embodiments of the present invention may provide a number of technical advantages. For example, according to one embodiment of the present invention, an architecture and a process are provided that offer a flexible system, which can easily accommodate any number of diverse surgeries. The obturator serves as a blunt instrument for directing an accompanying medical device. The obturator is blunt enough to avoid snagging a given tool or instrument in undesired locations; however, the obturator is pointed enough to give some guidance or directional capabilities for an accompanying device. This offers a high degree of accuracy in placing a given tool or instrument. In essence, the obturator eliminates the need for retrieving the initial item that allowed for a viable entrance (or pathway) into the surgical area.

In addition, the obturator is dissolvable: operating almost as a hologram in disappearing after a given time period so that if it breaks off and ventures into the bloodstream, it is of no consequence (i.e. no strokes or damage done to an end-organ). The time period is configurable, as the solubility of the obturator can be based on the chemicals used in the obturator, or it can be temperature-based. The obturator is further advantageous because it does not contaminate the interior of a given vessel or vein: such contact normally harms these sensitive items. Additional advantages are described herein with reference to corresponding FIGURES.

In another embodiment of the invention, the lens/device [being transparent] functions as a barrier to the contents of the hollow organ (e.g., aorta and blood), which allows the surgeon to carefully aim his device in a way that, on swift removal, allows the surgeon to insert/place that device into a hole that would otherwise be obscured by egressing blood (or gas, organ contents, etc.).

Certain embodiments of the present invention may enjoy some, all, or none of these advantages. Other technical advantages may be readily apparent to one skilled in the art from the following figures, description, and claims.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIG. 1 is a simplified schematic diagram illustrating an obturator to be used in a procedure in accordance with one embodiment of the present invention;

FIG. 2A is a simplified block diagram of an example set of potential shapes that a given obturator could take;

FIG. 2B is a simplified block diagram of an example tray for forming the shapes for the obturators of FIG. 2A;

FIGS. 3A-3C are simplified block diagrams illustrating an interface tool that is operable to assist a surgeon in placing a given device: either with or without the obturator;

FIGS. 4A-J are simplified schematic diagrams of another example operation that implicates the obturator;

FIGS. 5-10 are simplified schematic diagrams of additional example obturators and their associated components;

FIG. 11 is a top view of one example obturator; and

FIGS. 12-15 are simplified schematic diagrams of example coil elements that can be used in example embodiments of the present invention.

### DETAILED DESCRIPTION OF THE INVENTION

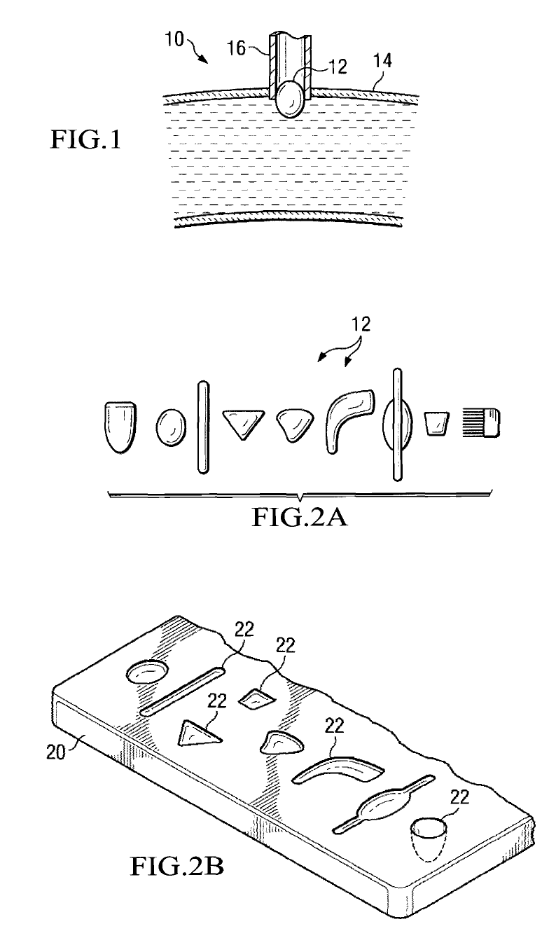

FIG. 1 is a simplified schematic diagram of an example vascular environment 10 in which an obturator 12 may be used during a given operation. In this first example, obturator 12 is being used in connection with an anastomosis between a tube (or a vein, or a vessel, etc.) 12 and a second conduit 14 (e.g., an aorta).

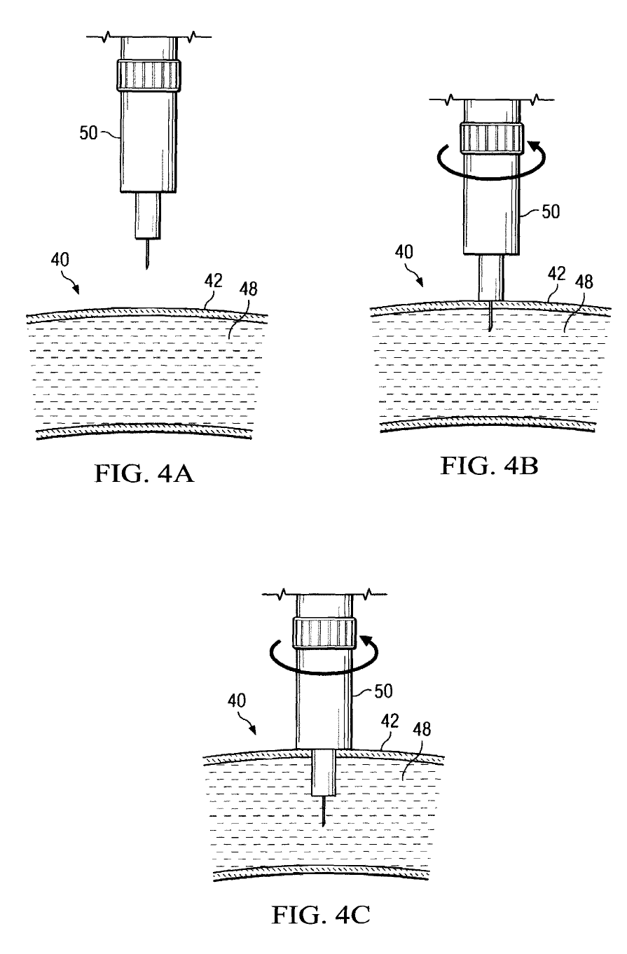

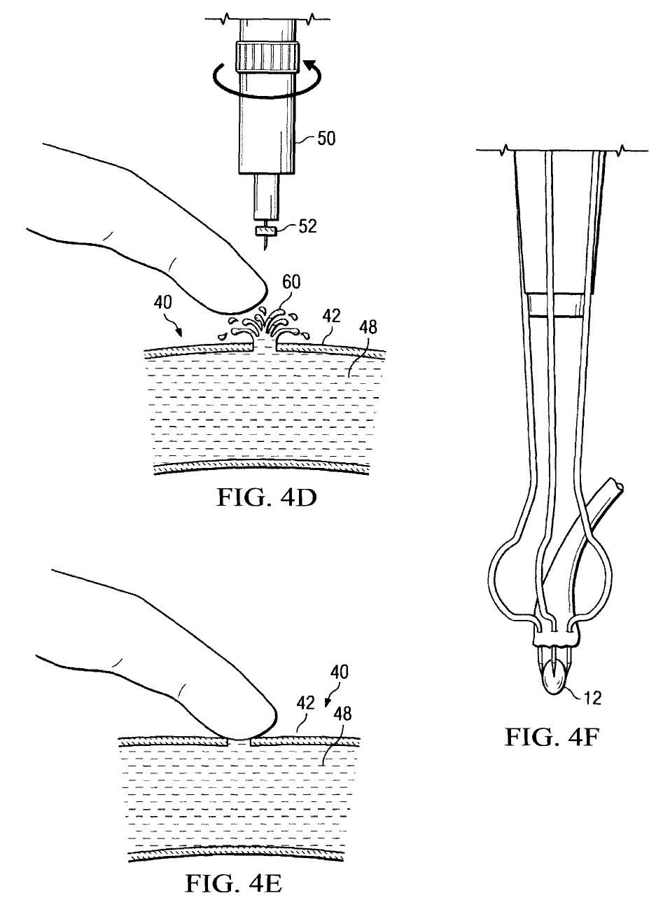

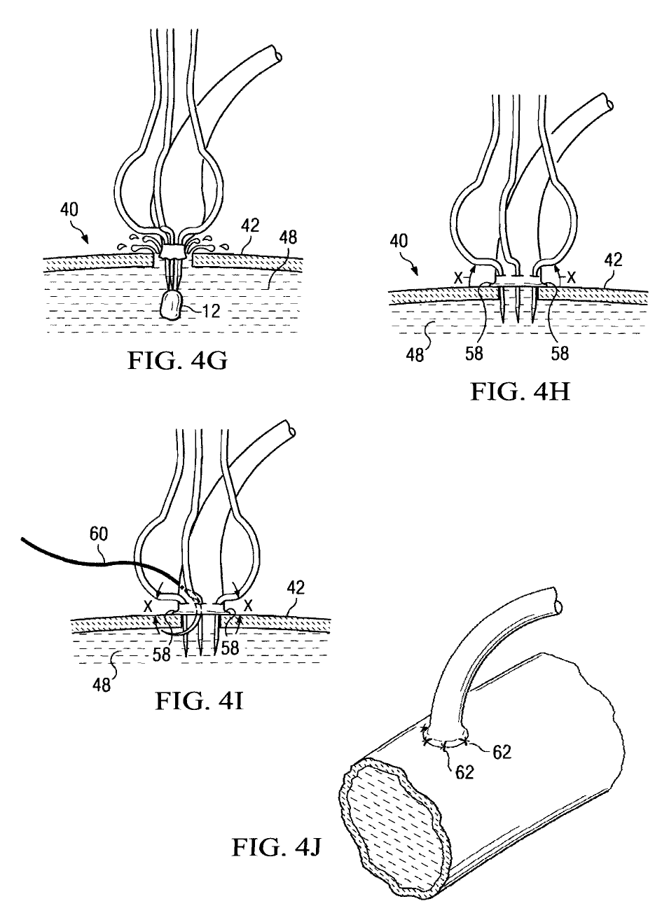

Note that the outer layer of the aorta is generally susceptible to trauma and inflammation. Specifically, devices such as cutters or vein-holding instruments (as illustrated in FIG. 4F) can easily become entangled while attempting to engage a hole or while making an incision. A high level of precision is necessary to avoid snagging in any area that surrounds the target area. In cases where entanglement does occur, a patient could suffer blood loss or, in other scenarios, a surgeon could inadvertently make an errant hole or a tear in surrounding tissue of the surgical area.

A few strategies may be employed to address this worrisome issue. One remedy, which is specific to a vein-holding apparatus inclusive of multiple needles, would allow all the needles to converge to a single point. This would allow a surgeon to effectively place a single needle, as opposed to a group of prickly ends, into a single destination. In this manner, the device becomes stream-lined: albeit in only one direction.

The present invention takes a different approach in offering an ideal obturator 12 that serves as a blunt instrument for directing an accompanying medical device. Obturator 12 is blunt enough to avoid snagging a given tool or instrument; however, obturator 12 is pointed enough to give some guidance or directional capabilities for an accompanying device. In this given example of FIG. 1, obturator 12 is being used in conjunction with a vein-holding apparatus such as that which is provided by Pending Patent Application having Ser. No. 11/084453 and Entitled: System and Method For Attaching a Vein, an Artery, or a Tube in a Vascular Environment [filed Mar. 18, 2005], which is hereby incorporated by reference herein in its entirety. In this example, the obturator is sized based on the vein's diameter: approximately 2-9 millimeters. However, this range has only been offered for example purposes and can certainly be changed to accommodate other arrangements based on particular needs.

Obturator 12 can be dissolvable: operating almost as a hologram in disappearing after a given time period. The time period is configurable, as the solubility of obturator 12 can be based on the chemicals used in obturator 12 or it can be based on how frozen obturator 12 is made to be (or its intrinsic temperature). Obviously, this frozen rigidity will dissolve when exposed to other external elements, such as a person's blood, or the ambient temperature of the operation room.

Obturator 12 is further advantageous because it does not contaminate the interior of a given vessel or vein: such contact normally harms these sensitive items. Obturator 12 can be employed such that harmful touching is effectively avoided. Obturator 12 may be constructed of a gas, water, sugar, clotted blood, a gelatinous material, a protein, a saline solution, dry-ice, or any suitable combination thereof. Obturator 12 would dissolve in a time that would be short enough to minimize the potential for having a stroke. For example, the total time to dissolve may be about 2:00 minutes. However, obturator 12 could be used to offer any suitable time period for dissolution.

In essence, obturator 12 can allow a surgeon to gain entrance into a given organ, while effectively eliminating the original, entrance obturator. The initial obturator effectively disappears: without any additional effort from the surgeon. This could be important for gaining access to the apex of the heart, or in the field of robotics, or in remote vascular procedures, or in instances where something is propped up or glued or fixed by the surgeon. The common theme here is an elimination of the need for retrieving the initial item that allowed for an entrance into the surgical area. The advantage of obturator 12 is that it achieves a stream-lined effect in a first direction, but does not suffer from a difficult removal in the second direction (i.e. during exit).

FIG. 2A is a simplified block diagram of an example set of potential shapes that a given obturator could take. Some of these include comb shapes (which would prohibit a predetermined amount of blood flow from exiting a given area, while potentially allowing legs of given device to penetrate some of the seams of the comb), bullet shapes, triangular shapes, etc. Note that these shapes are not exhaustive and are only being provided in hopes of offering some example configurations to be used in accordance with the present invention.

FIG. 2B is a simplified block diagram of a tray 20 for forming the shapes for the obturator of FIG. 2A. A set of molds 22 are provided to illustrate how the obturator may be formed. Tray 20 may simply be placed in a refrigerating compartment (or other suitable cooling mechanism) such that the obturator becomes solid. Note that most hospital rooms that are used to accommodate surgical procedures include a small water freezer or slush machine that could certainly be used to fulfill this purpose.

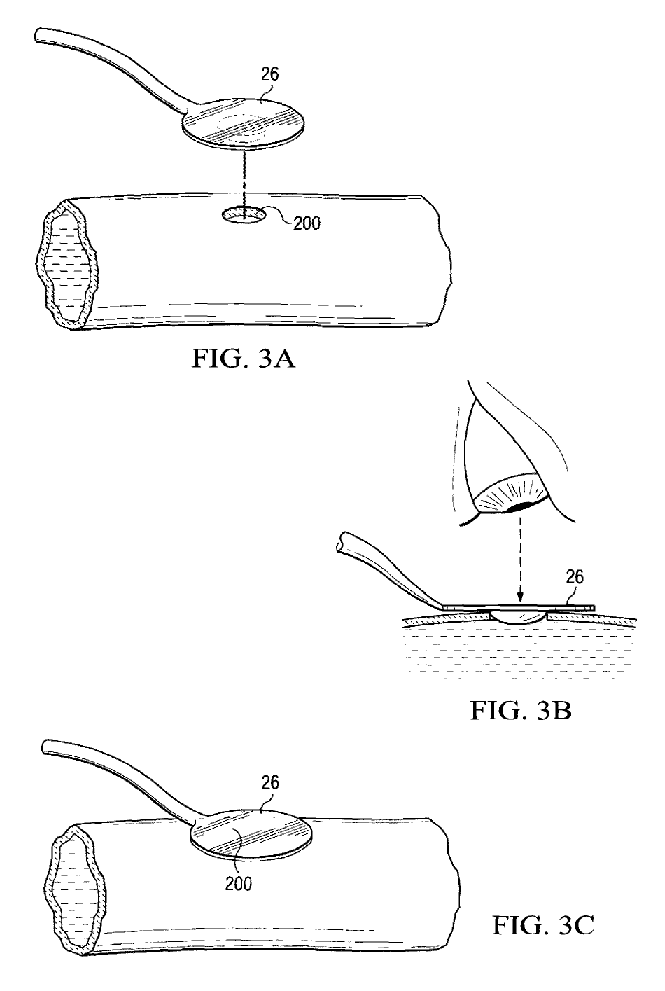

FIGS. 3A-3C are simplified block diagrams illustrating an interface tool 26 that is operable to assist a surgeon in placing a given device: either with or without obturator 12. As illustrated, interface tool 26 may be transparent (or oblique or opaque in other embodiments) and may include a magnification element such that a surgeon may see the underlying element in greater detail. Interface tool 26 may include a convex portion 200 (this may or may not include the magnification element). In cases where there is a hole provided in the underlying issue, convex portion 200 may operate to seat interface tool 26 into the surface. In this sense, a surgeon can feel his way into an opening, as convex portion 200 engages the surface area of the targeted location and is suitably secured thereon. In action, interface tool 26 may be used to suppress blood flow (or other fluids) from escaping from the underlying tissue. In this sense, interface tool 26 maintains the interior pressure and may be used as a stop-gap until the attending surgeon is prepared to address the targeted area or the incised hole.

Before proceeding further, for purposes of teaching and discussion, it is useful to provide some overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

The basic tenets of vascular operations allow us to appreciate that surgeries can be highly invasive and, therefore, present a number of risks. For example, existing devices that are used in many current operations can be difficult to control, as the surgeon must have extraordinary dexterity in manipulating the requisite components. In other scenarios, devices may offer benefits associated with ease of use; but these devices are generally flawed because they often improperly violate the inside of the aorta or facilitate entanglement problems. In the context of bypass procedures, contact with the aorta should be avoided because plaque or any other (potentially friable) detrimental element can flake off and become dislodged in other anatomical locations.

Obturator 12 overcomes the aforementioned deficiencies, as well as others, in providing an optimal solution for a physician who is relegated the difficult task of performing a vascular procedure. Obturator 12 is intuitive in that it guides an accompanying device and, further, offers exceptional flexibility and adaptability for a physician. In addition, obturator 12 offers enhanced accuracy for a surgeon who must make precise incisions (e.g., in the aortic wall, or the apex of the heart, etc.). No longer would a surgeon have to guess or to estimate the location of a given hole in an organ or in a piece of tissue. Additionally, such an instrument is minimally invasive because removal of obturator 12 (because of its solubility) is no longer necessary.

FIGS. 4A-J illustrate how a hole may be cut in an aortic wall 40. FIG. 4A includes a cutter 50, which may be used to make a small incision into the aorta. FIG. 4A also illustrates blood flow 48, as well as an initial aortic layer 42, which is penetrated by cutter 50. The aortic cutter 50 is used to make a uniform hole in the aorta for attaching the vein bypass graft.

FIG. 4B illustrates cutter 50 being engaged, whereby layer 42 is pierced. FIG. 4C illustrates cutter 50 being suitably positioned within aortic wall 40 just before the hole is actually cut. FIG. 4D illustrates the hole that was created and the resulting blood flow. Cutter 50 may be automatic or mechanical, as its rotation can produce a uniform incision in either case. Because of the blood flow, the surgeon can simply put his finger over the hole prior to plugging the hole with the vein, as is illustrated by FIG. 4E. FIG. 4F illustrates how obturator 12 can be grasped by a medical instrument such that obturator 12 can be used to guide the device to a targeted area: free from snagging in undesirable locations.

FIGS. 4G through 4J illustrate a transfer operation in which the vein is used to fill the void in the aortic wall. This process involves the proximal attachment, whereas the distal attachment involves a separate procedure. FIG. 4G illustrates how the hole is plugged with a medical device, which now includes a loaded vein. Note that obturator 12 is within the tissue, as it begins to dissolve and erode from its original form. At this stage, the legs of the medical device are somewhat close together and the vein is compressed. A handle can then be pulled or pushed such that the legs are somewhat extended and the vein is, thereby, expanded, as is shown in FIG. 4H. Obturator 12 is completely dissolved at this point such that the surgeon does not need to worry about retrieving the original obturator 12, which facilitated entry into the target location.

Note that there is some pressure in the vein such that, once suitably positioned, the vein will inflate to capacity. FIG. 4H further illustrates the design consideration that is given to the legs. An angle ‘X’ is created between aortic wall 42 and the legs. This enables a lip 58 of the vein to be produced, which can further enable the suturing process.

FIG. 4I illustrates the seal that is formed, whereby the vein is taut and ready to be sutured to the aorta. FIG. 4I illustrates a first suture 60 being positioned around the vein. Alternatively, a clip or some other mechanism may be used to join these two interfaces. Sutures may be ideal because they are benign and because they offer a simple protocol for the surgeon.

FIG. 4J illustrates a series of sutures 62 having been properly placed around the bypass vein. The suturing operation was assisted by the design of the legs, whereby such a fastening procedure provides a reliable means for securing the vein to the aortic wall. Utilizing such a suturing approach achieves some level of comfort for the tending surgeon, as suturing represents a customary method of accomplishing such an attachment. Thus, obturator 12 has provided a virtually seamless entry and exit for the surgeon at the targeted surgical site.

Note that any of the previously discussed materials could be included in a given kit, which could ostensibly be provided to a physician who is responsible for performing a procedure. A basic kit could include a given medical device, obturator 12, and interface element 26. The kit could also include one or more closures for suturing or affixing the vein or tube. Any of these components may be manufactured based on particular specifications or specific patient needs. The present invention contemplates considerable flexibility in such components, as any permutation or modification to any of these elements is clearly within the broad scope of the present invention.

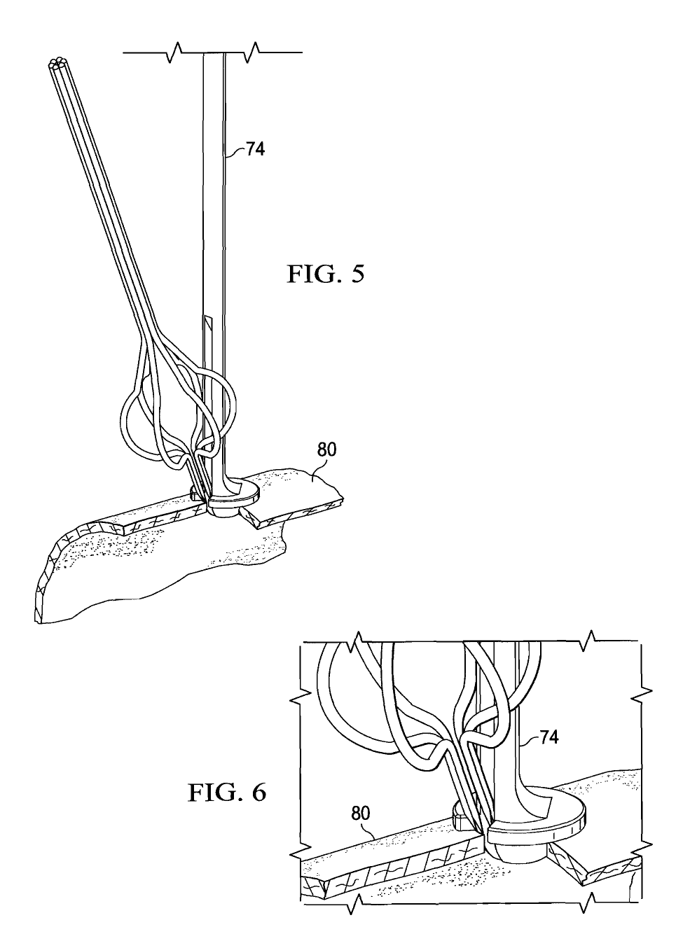

FIGS. 5-10 are simplified schematic diagrams of additional example obturators and their associated components for guiding element into a targeted region. More specifically, FIG. 5 depicts an obturator 74, which is shown engaging a cut hole in an aorta (not shown for purposes of simplification). Obturator 74 includes a long shaft that can help in guiding any given instrument to a targeted region. For example, a tip of an instrument could be directed down the shaft and toward its intended target. Note that as used in FIGS. 5-14, the term ‘tip’ is meant to encompass any contact point of a medical instrument, tool, or device. As such, the ‘tip’ could be dull or blunt itself, as obturator 74 still has value for applications that seek to guide these elements to their ultimate destinations.

A knob of obturator 74 engages a given hole. A flange element 80 of obturator 74 extends beyond the knob onto an outside surface of the aorta to minimize bleeding that may occur in a vascular procedure.

In one example, a V-shaped groove of obturator 74 collects sharp needle tips from a tool. This collecting feature helps introduce needles into a cut hole of an aorta, while lessening the tendency to catch sharp tips on the surrounding tissue. In one example, one function of the knob can be to keep the obturator securely over the hole. The v-groove of the obturator can help stabilize and guide the needle tips on a tool (e.g., the vein holding device). Thus, the groove can stabilize a tool laterally on the surface of the aorta, or any other suitable surface. FIG. 6 merely magnifies the embodiment of FIG. 5.

The angle of obturator 74 (with reference to the aortotomy) is important. In one embodiment, a straight (perpendicular) shaft can be used. In other embodiments, to attenuate leaking concerns, the angle can be changed considerably. Thus, obturator 74 can have variable angles: ranging from perpendicular to virtually horizontal. In still another embodiment, the working angle is about 30 degrees.

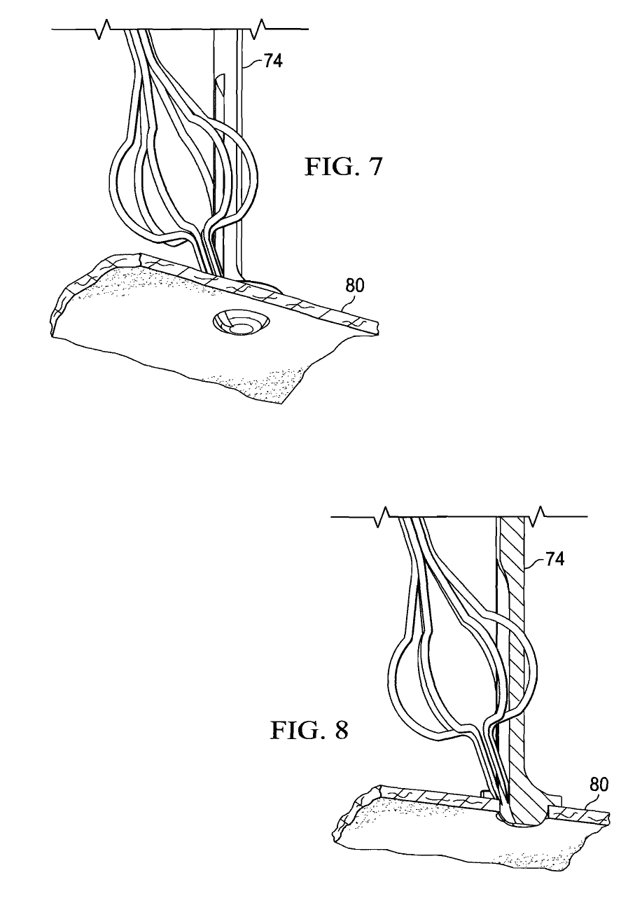

FIG. 7 depicts a view of obturator 74 from inside a given cavity or conduit (e.g., an aorta). FIG. 7 also illustrates the knob of obturator 74 engaging a cut hole in the conduit (e.g., an aorta) . FIG. 8 shows a sectional view of obturator 74. A chamfer feature of the knob can provide room for the needles of a device to enter the conduit or tissue (e.g., an aorta) at an angle without bumping into the knob.

The shape of the contact interface is significant. In some instances, this contact interface of obturator 74 can be the knob described, a bulging half-circle, a type of curved element, or at least a convex portion that operates to facilitate seating of any of the obturators described herein. In still other embodiments, the contact interface is a square, a rectangle, a diamond, or a triangle shape.

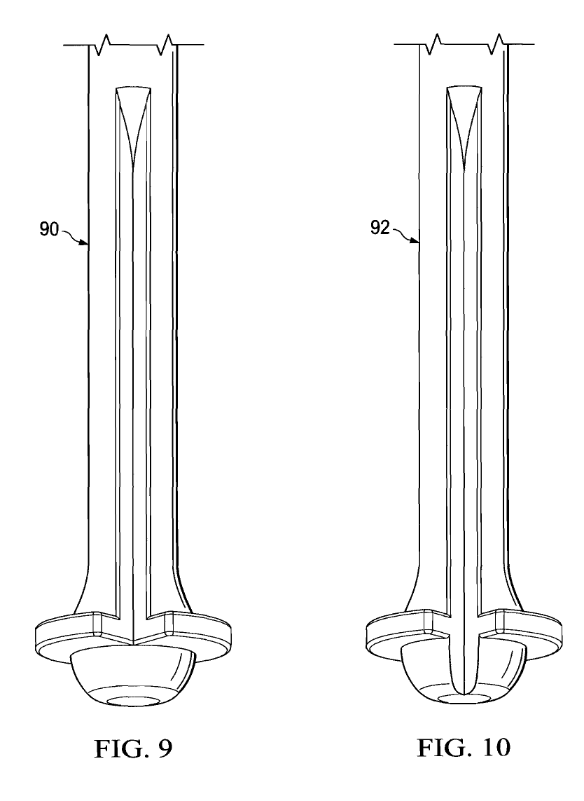

FIG. 9 illustrates another obturator 90, which includes a groove. The grove could be V-shaped in one embodiment to help in guiding a device or for needle collecting purposes, where a tool has a multitude of legs. In this instance of FIG. 9, the V-shaped groove does not extend into the knob of obturator 90. Such a configuration can potentially minimize bleeding, but may also require a point (or needles) to slide between the knob and the cut portion of the tissue (e.g., the wall of the aorta).

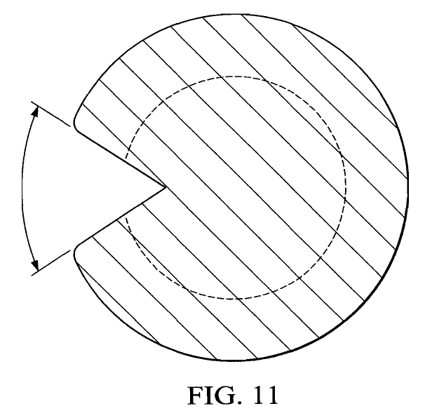

FIG. 10 illustrates an obturator 92 that includes a V-shaped groove that extends into the knob of the tool. This configuration may lessen the chance of sharp tips of a tool catching the surrounding tissue. Note that for FIGS. 9-10, both obturators could include a chamfer on the knob (i.e., the obturator's knob may or may not be chamfered). In a general sense, these elements in FIGS. 9-10 are offering a rigid plug obturator. An additional embodiment of these obturators has the V-shaped groove designed deeper so it cuts into the cylinder that occupies the hole (e.g., an aortic hole). This may allow some room for the tips to get in, but may result in some blood coming from the pressurized aorta. Note that, in broad terms, any of the obturators shown in FIGS. 5-10 can include any suitable bottom portion that is substantially blunt such that it exhibits a snag-resistant property. FIG. 11 is a top view of one example obturator

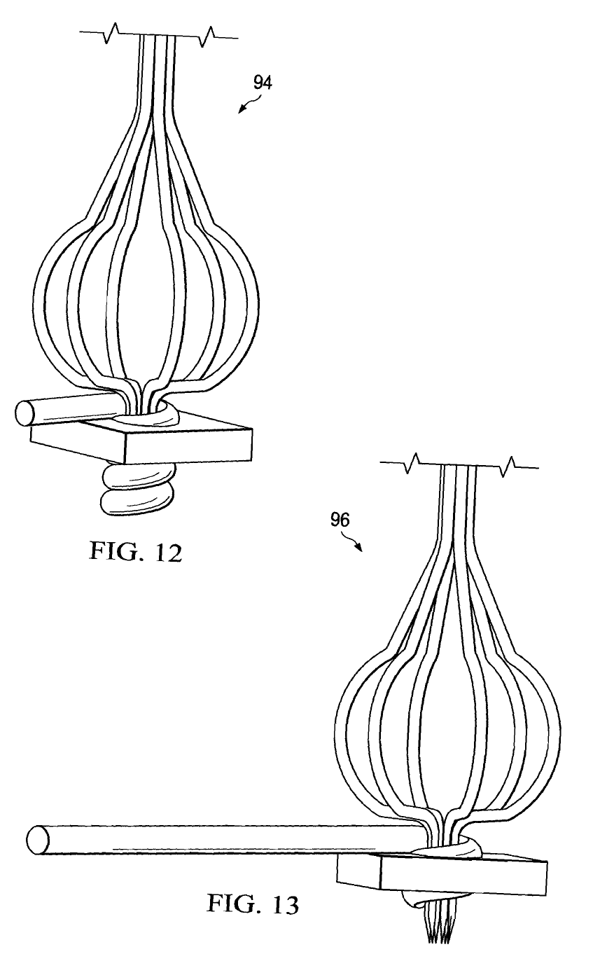

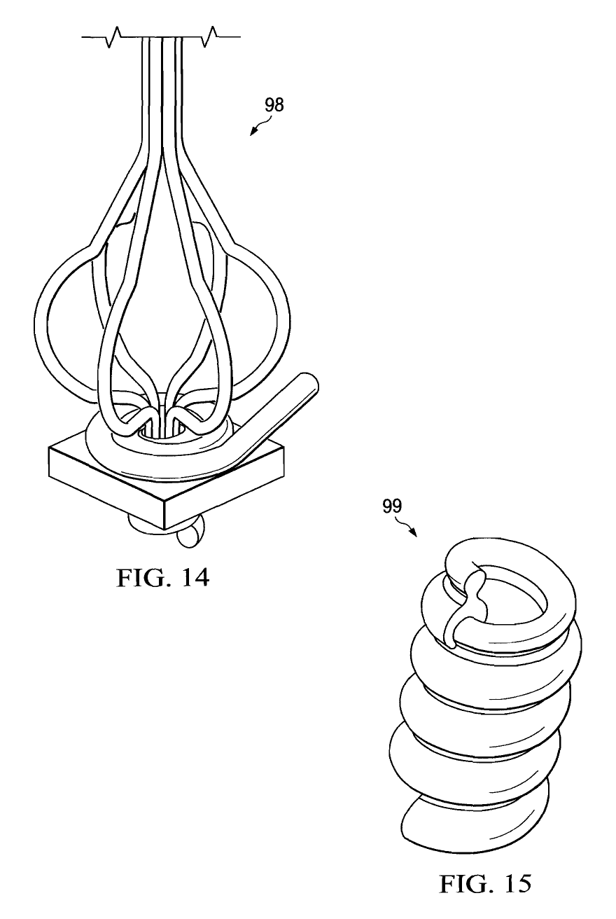

FIGS. 12-15 are simplified schematic diagrams of example coil elements that can be used with some embodiments of the present invention. FIG. 12 illustrates a straight coil assembly 94 in accordance with an example embodiment of the present invention. FIG. 13 illustrates an example flared coil assembly 96 that can be used with example embodiments of the present invention. FIG. 14 is a simplified schematic diagram of an uncoiling operation associated with example activities of the present invention. The uncoiling activity is represented generally at 98. FIG. 15 is a simplified schematic diagram illustrating a link coil beading 99 in accordance with another example embodiment of the present invention.

In operation of an example scenario implicating FIGS. 12-15, a vein-holding apparatus (such as that referenced earlier in this Specification) may be used in conjunction with these elements. For example, after loading of the vein onto the system's needles, the needles could be drawn together (for example, by pulling back a plunger-like mechanism on the handle). A helical coil (such as that depicted, for example) could be placed over the needle bundle. The coil could be made from a soft [solid] plastic cord and/or heat-formed into shape. Other materials could certainly be used and are clearly within the broad teachings of the present invention.

The handle plunger could subsequently be pushed (for example, to attempt to expand the needles of the vein-holding device) . The coil assembly depicted in FIGS. 12-15 could prevent expansion of the needles. Upon coil removal (for example, as the coil is unwound (meaning: either through a simple rotation where the coil is integral or through pulling a portion of the coil away from itself where the coil is designed as a wrapping mechanism), expansion could occur immediately. Alternatively, the material used in the coil could engender a slow expansion, where a resistive force is applied to the confined needles. Once the needles of the device are covered by the coil, a hole may be cut into the aorta.

The coil can be a hybrid of the free, loose coil and the linked together coil. One example includes forming the coil (e.g., a simple round cross section bead) with a process that results in neighboring coils lightly adhering to each other. This adherence allows the coil to maintain a strong cylindrical structure when containing needles and, further, when sliding/guiding the assembly into a hole. However, such an embodiment can be uncoiled by propagating a tear of the slight bond to unwind the coil. Other permutations and derivations of such an example structure are clearly within the scope of the present invention.

The loaded system, inclusive of the harvested vein (with shielded needles) can be positioned into the hole of the aorta to seal the closure. The tail of the coil can be pulled (for example, to uncoil the device) until the needles are free and the system expands in the hole of the aorta for subsequent suturing.

The illustrations of FIGS. 12-15 depict a simple (cylindrical) coil that shields needles penetrating through a cut hole in the aorta. The coil is slightly flared at its top to further minimize fluid escape (e.g. squirting). Other illustrations demonstrate the coil being removed (just prior to the needles of a vein-holding device being freed). Also provided is an illustration of a coil element, where neighboring coils are not free to spread apart (but could still be rotated to uncoil and free the needles). This may be an advantage to not allow coils to shift laterally and, furthermore, it may be desirable to offer a sturdy shielding structure. Thus, in such an embodiment an integral, unitary coil element is defined. In other embodiments, the coil is a combination in which a portion of the coil is integral and fixed and the other half of the coil includes rings capable of being unwound.

Note that the shape and dimensions of the coil can be easily changed without departing from the concepts of present invention. For example, a square design could easily be accommodated by the teachings of the present invention. Along similar reasoning, triangular and oval-shaped designs could be used to help control the needles, as they are being used in a given vascular procedure. There are countless possibilities that are clearly within the broad scope of the present invention. Moreover, the coil design could be such that the depth at which the needles penetrate (or are seen in) the aortic wall is considerably different from that which has been depicted by FIGS. 12-15.

It should also be noted that although FIGS. 12-15 have been discussed with reference to a certain device being used in a vascular environment, these elements of FIGS. 12-15 can be used independently and not necessarily with a vein-holding apparatus as described herein. Moreover, other applications that need (or desire to have) a harnessing of tips or of sharp objects could certainly benefit from the coil elements described and depicted in FIGS. 12-15.

It is important to note that the stages and steps in the preceding FIGURES illustrate only some of the possible scenarios that may be executed by, or within, the architecture of the present invention. Some of these stages and/or steps may be deleted or removed where appropriate, or these stages and/or steps may be modified or changed considerably without departing from the scope of the present invention. In addition, a number of these operations have been described as being executed concurrently with, or in parallel to, one or more additional operations. However, the timing of these operations may be altered considerably. The preceding example flows have been offered for purposes of teaching and discussion. Substantial flexibility is provided by the proffered architecture in that any suitable arrangements, chronologies, configurations, and timing mechanisms may be provided without departing from the broad scope of the present invention.

Note also that the example embodiments described above can be replaced with a number of potential alternatives where appropriate. The processes and configurations discussed herein only offer some of the numerous potential applications of the device of the present invention. The elements and operations listed in FIGS. 1-15 may be achieved with use of the present invention in any number of contexts and applications. Accordingly, suitable infrastructure may be included within a given system (or cooperate with obturator 12 in some fashion) to effectuate the tasks and operations of the elements and activities associated with managing a procedure.

Although the present invention has been described in detail with reference to particular embodiments in FIGS. 1-15, it should be understood that various other changes, substitutions, and alterations may be made hereto without departing from the sphere and scope of the present invention. For example, although the preceding FIGURES have referenced a number of components as participating in the numerous outlined procedures, any suitable equipment or relevant tools may be readily substituted for such elements and, similarly, benefit from the teachings of the present invention. These may be identified on a case-by-case basis, whereby a certain patient may present a health risk factor while another (with the same condition) may not. Hence, the present tool may be designed based on particular needs with particular scenarios envisioned.

It is also imperative to note that although the present invention is illustrated as implicating several example procedures, this has only been done for purposes of example. The present invention could readily be used in virtually any procedure where an obturator would be beneficial and, accordingly, should be construed as such. The present invention may easily be used to provide a viable vascular management solution at various locations of the mammalian anatomy, which are not necessarily illustrated by the preceding FIGURES.

Numerous other changes, substitutions, variations, alterations, and modifications may be ascertained to one skilled in the art and it is intended that the present invention encompass all such changes, substitutions, variations, alterations, and modifications as falling within the spirit and scope of the appended claims. In order to assist the United States Patent and Trademark Office (USPTO) and additionally any readers of any patent issued on this application in interpreting the claims appended hereto, Applicant wishes to note that the Applicant: (a) does not intend any of the appended claims to invoke paragraph six (6) of 35 U.S.C. section 112 as it exists on the date of filing hereof unless the words “means for” are specifically used in the particular claims; and (b) does not intend by any statement in the specification to limit his invention in any way that is not otherwise reflected in the appended claims.

What is claimed is:

1. An apparatus, comprising:
a coil that receives one or more tips of an instrument, wherein an interior of the coil interfaces with the tips and the coil and the tips can be positioned together in a hole cut in a surface of tissue, the coil operable to be unwound such that a portion of the tips is revealed as the coil is unwound.

2. The apparatus of claim 1, wherein the instrument is a vein-holding apparatus, and wherein the tips hold a vein to be sutured at the hole.

3. The apparatus of claim 1, wherein the coil is helically shaped.

4. The apparatus of claim 1, wherein the coil is shaped as a plastic cord that is wound around the tips.

5. The apparatus of claim 1, wherein the interior of the coil is integral such that a unitary cavity is defined by the coil.

6. The apparatus of claim 1, wherein the coil includes a tail element that is pulled to unwind the coil incrementally.

7. The apparatus of claim 6, wherein the tail element can be pulled until the tips are exposed from the coil.

8. The apparatus of claim 1, wherein the coil is flared at a top portion of the coil to minimize fluid escape.

9. The apparatus of claim 1, wherein the coil includes a first portion in which one or more rings can be wound and unwound, and wherein the coil includes a second portion in which one or more rings of the coil are fixed and cannot be wound or unwound.

10. A method, comprising:
receiving one or more tips of an instrument via a coil, wherein an interior of the coil interfaces with the tips; and
positioning the coil and the tips together in a hole cut in a surface of tissue, the coil operable to be unwound such that a portion of the tips is revealed as the coil is unwound.

11. The method of claim 10, wherein the instrument is a vein-holding apparatus, and wherein the tips hold a vein to be sutured at the hole.

12. The method of claim 10, wherein the coil is helically shaped, and wherein the coil is shaped as a plastic cord that is wound around the tips.

13. The method of claim 10, wherein the coil includes a first portion in which one or more rings can be wound and unwound, and wherein the coil includes a second portion in which one or more rings of the coil are fixed and cannot be wound or unwound.

14. The method of claim 10, wherein the interior of the coil is integral such that a unitary cavity is defined by the coil.

15. The method of claim 10, wherein the coil includes a tail element that is pulled to unwind the coil incrementally.

16. The method of claim 15, wherein the tail element can be pulled until the tips are exposed from the coil.

17. The method of claim 10, further comprising:
loading a vein onto the instrument, which secures the vein; and
positioning the vein in an aortic hole.

18. An apparatus, comprising:
a coil that receives one or more tips of an instrument, wherein an interior of the coil interfaces with the tips and the coil and the tips can be positioned together in a hole cut in a surface of tissue, the coil operable to be unwound such that a portion of the tips is revealed as the coil is unwound, wherein the coil is helically shaped and includes a tail element that is pulled to unwind the coil incrementally, wherein the tail element can be pulled until the tips are exposed from the coil.

19. The apparatus of claim 18, wherein the instrument is a vein-holding apparatus, and wherein the tips hold a vein to be sutured at the hole.

20. The apparatus of claim 18, wherein the coil is flared at a top portion of the coil to minimize fluid escape.
